(19) United States
(12) Patent Application Publication
(10) Pub. No.: US 2020/0188252 A1
(43) Pub. Date: Jun. 18, 2020

(54) TWO-COMPONENT HAIR CARE PRODUCT FOR PREPARING AN EMULSION FOR GROOMING HUMAN HAIR

(71) Applicant: Henkel AG & Co. KGaA, Duesseldorf (DE)

(72) Inventors: RENE KROHN, Norderstedt (DE); ERIK SCHULZE ZUR WIESCHE, Hamburg (DE)

(73) Assignee: Henkel AG & Co. KGaA, Duesseldorf (DE)

(21) Appl. No.: 16/716,108

(22) Filed: Dec. 16, 2019

(30) Foreign Application Priority Data
Dec. 18, 2018 (DE) 10 2018 222 145.6

(51) Int. Cl. A61K 8/31; A61K 8/34; A61K 8/37; A61K 8/42; A61K 8/06; A61Q 5/12

(52) U.S. Cl. CPC A61K 8/31; A61K 8/342; A61K 8/37; A61K 2800/262; A61K 8/06; A61Q 5/12; A61K 2800/882; A61K 8/42

(57) ABSTRACT

The present disclosure relates to an anhydrous carrier as a first component and an aqueous carrier as a second component, wherein the anhydrous carrier contains a) a branched or unbranched alkane, b) a branched or unbranched fatty alcohol and c) an alkyl ester, a N,N-dialkyl amide or an ether, and the aqueous carrier comprises a cationic tenside.

### CROSS-REFERENCE TO RELATED APPLICATION

This application claims priority to German Patent Application No. 10 2018 222 145.6, filed Dec. 18, 2018, which is incorporated herein by reference in its entirety.

### TECHNICAL FIELD

The present disclosure relates to the field of preparing hair care products, particularly hair conditioning agents in cosmetics.

### BACKGROUND

The present disclosure relates particularly to an anhydrous carrier as a first component and an aqueous carrier as a second component, wherein the anhydrous carrier contains a) a branched or unbranched alkane, b) a branched or unbranched fatty alcohol and c) an alkyl ester, a N,N-dialkyl amide or an ether, and the aqueous carrier comprises a cationic tenside.

### BRIEF SUMMARY

Cosmetic products for cleaning and/or grooming hair have been known for a long time and are regularly improved and/or adapted to the changing needs of consumers. For example, consumers expect that a modern hair grooming product or conditioner will clean and smooth their hair, make it easier to comb, soft, shiny, easy to style, as well as moisturising, combating dandruff and lending it more volume, and more. Many active substances are known which can be used in such a cosmetic product to satisfy one or more of the requirements listed above. However, the presence of large quantities of different substances can destabilise the hair cosmetic, and for this reason manufacturers usually attempt to satisfy as many of the aforementioned requirements as possible using a small number of compatible substances.

A large number of different components may have the effect of limiting freedom of formulation during the development of cosmetic products, because components interact with each other or interfere with the structure. For example, substances may upset the desired structure of an emulsion, although it is precisely the desired structure of the emulsion that may be associated with advantageous cosmetic effects. Components of an aqueous phase and components of a fatty phase may also have properties that hinder the formation of emulsions. Thus even the emulsion formation, that is to say the manufacture of the emulsion, affects the formulation. The freedom of formulation is further limited by the requirement to ensure good biodegradability. The task of complying with the totality of all requirements to a satisfactory degree presents developers with considerable challenges.

The problem addressed by the present disclosure consists in providing a cosmetic product, particularly a hair grooming and/or conditioning agent constituting an emulsion that can be manufactured easily. The problem addressed by the present disclosure consisted more particularly in providing a cosmetic product with the capability to offer the maximum possible freedom of formulation. A further problem addressed by the present disclosure consisted in providing a cosmetic product that can be managed with ease by consumers.

The problem is solved with the object of Claim 1. Accordingly, a first object of the present disclosure is a two-component hair care product comprising separately from each other an anhydrous carrier as a first component and an aqueous carrier as a second component, the anhydrous carrier contains
a) a branched or unbranched alkane having a chain length from C10 to C30, preferably from C12 to C24, more preferably from C14 to C20,
b) a branched or unbranched fatty alcohol having a chain length from C10 to C22, preferably C12 to C18, more preferably from C14 to C16, and
c) an alkyl ester, a N,N-dialkyl amide or an ether, wherein the total carbon atomic number in the alkyl ester, the N,N-dialkyl amide or the ether is in the range from about 6 to about 30, preferably from about 8 bis about 22, more preferably from about 10 to about 16, most preferably from about 12 to about 14,
   and the aqueous carrier comprises a cationic tenside including an organic radical with a linear or branched chain length of C12 to C30, preferably C14 to C26, more preferably C16 to C22.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Surprisingly, it was found that the two components, i.e. the aqueous carrier and the anhydrous carrier, are readily mixable with each other to form an emulsion. A number of advantageous effects are associated with this. On the one hand, constituents may be present in the differing components which—if they are present in the same composition—interact in such a way that over a prolonged period they present a risk of unfavourable effects. This therefore increases the freedom of formulation, since constituents in the components do not come into contact with each other until shortly before application. The fact that constituents of the components are incompatible over a prolonged period is only a minor consideration. On the other hand, with the object as contemplated herein users have at their disposal the capability of using a stable emulsion: Because of the ease with which the aqueous carrier and the anhydrous carriers can be combined to yield an emulsion, the user can mix the components shortly before the application and immediately obtain an emulsion which is stable until it is applied. Moreover, the user is also in possession of an experience product. It may be used to create an application-ready cosmetic product according to the user's needs. A two-component kit enhances confidence in the performance capability of the product.

The two-component hair care product is preferably not a hair dye or bleaching agent. Apart from the points discussed previously, it is preferable if the two-component hair care product as contemplated herein does not contain any oxidising agents, no direct dyes and no oxidation dye precursors for dyeing keratinous fibres, particularly human hair.

The first carrier of the composition as contemplated herein should be anhydrous, more particularly substantially anhydrous. The person skilled in the art understands an anhydrous carrier to be a composition to which no water is added. Given the fact that raw materials generally are not entirely water-free, the anhydrous carrier contains a small quantity of water. Moreover, products always absorb a certain degree of moisture. A preferred anhydrous carrier is substantially free of water. More preferably, the anhydrous carrier comprises from 0 to about 3 wt % water, more preferably from about 0.1 to about 2 wt %, most preferably from about 0.2 to about 1 wt % relative to the total weight of the carrier which is described as anhydrous.

As a first constituent, the anhydrous carrier of the two-component hair care product as contemplated herein comprises a branched or unbranched alkane. This is essentially selected such that it is fluid at a temperature of about 20° C. and a pressure of about 10⁵ Pa. Consequently, the alkanes with a higher number of carbon atoms are mostly branched alkanes, whereas the alkanes with a lower number of carbon atoms may be linear or branched alkanes. The alkane essentially constitutes the organic basis of the anhydrous carrier.

Preferred alkanes are thus liquid paraffin oil, isoparaffin oil or synthetic hydrocarbons. One product that is available commercially may be 1,3-di-(2-ethyl-hexyl)-cyclohexane (Cetiol® S). Most particularly preferred is the alkane dodecane or isododecane.

As a second constituent, the anhydrous carrier of the two-component hair care product as contemplated herein comprises a fatty alcohol. Fatty alcohols that are suitable for use are monovalent—that is to say fatty alcohols with one or two OH groups—saturated, mono- or polyunsaturated, branched or linear fatty alcohols. Fatty alcohols that are usable for the purposes of the present disclosure include for example decanol, dodecenol, decenol, octadienol, dodecadienol, decadienol, oleyl alcohol, erucyl alcohol, ricinol alcohol, lauryl alcohol, myristyl alcohol, cetearyl alcohol, capric alcohol, linoleyl alcohol and linolenyl alcohol. Particularly preferred are cetearyl alcohol and myristyl alcohol. Particularly preferred fatty alcohols are also Coconut Alcohol (INCI), Cetearyl Alcohol (INCI), Stearyl Alcohol (INCI), Cetyl Alcohol (INCI), Myristyl Alcohol (INCI), Lauryl Alcohol (INCI) and Decyl Alcohol (INCI). Fatty alcohol blends representing a mixture of various fatty alcohols are also usable as contemplated herein.

As a third component, the anhydrous carrier of the two-component hair care product as contemplated herein contains at least one compound chosen from an ether, an alkyl ester and a N,N-dialkyl amide. According to a preferred embodiment, the two-component hair care product contains as constituent c) an ether which is an alkoxylated fatty alcohol which preferably has the formula HO(C₃H₆O)ₙCₘH₂ₘ₊₁, in which n is a number from about 1 to about 6, preferably from about 2 to about 4, and m is a number from about 5 to about 14, more preferably from about 6 to about 12, most preferably from about 8 to about 10. In particular, the ethers used as contemplated herein are particularly suitable for solving the problem that is addressed by the present disclosure.

As a most particularly preferred ether, the anhydrous carrier contains PPG-3-Caprylyl Ether (INCI), a caprylyl ether which includes three propylene glycol groups.

According to a further preferred embodiment, the two-component hair care product contains as constituent c) an alkyl ester which is a fatty acid ester. More preferably, the fatty acid ester is a glycerol triester which has identical or different acyl groups. More preferably still, the acyl groups each include from about 5 to about 11, preferably from about 7 to about 9, carbon atoms.

A most particularly preferred glycerol triester is Capric/Caprylic Triglyceride (INCI).

Alternatively, the two-component hair care product may contain as constituent c) an alkyl ester which is a fatty acid ester that is preferably a product of esterification from a C4 to C14, preferably C6 to C10, fatty acid and a C4 to C14, preferably C6 to C10, fatty alcohol.

A further most particularly preferred alkyl ester is Coco Caprylate (INCI).

According to a further preferred embodiment, the two-component hair care product contains as constituent c) a N,N-dialkyl amide that is a fatty acid amide which is preferably a N,N-dimethyl-carboxylic acid amide having a C8 to C18, preferably a C10 to C16, more preferably a C12 to C14 acyl group. Alkyl amides in which the amide nitrogen contains two alkyl groups, particularly methyl groups are used as N,N-dialkyl amides. The amide has an acyl group with 8 to 18, preferably 10 to 16, more preferably 12 to 14 carbon atoms. Particularly preferred are Dimethyl Lauramide and Dimethyl Myristamide and mixtures thereof.

The N,N-dialkyl amide is exceedingly preferably selected from mixtures of Dimethyl Lauramide and Dimethyl Myristamide (Dimethyl Lauramide/Myristamide (INCI)).

The use of the most preferred constituents c) has a positive effect on the problem the present disclosure is intended to address.

As contemplated herein, the aqueous carrier of the two-component hair care product contains a cationic tenside which comprises an organic radical with a linear or branched chain length from C12 to C30, preferably C14 to C26, more preferably from C16 to C22. Besides the cationic group, the cationic tenside thus contains one or also more organic radicals, but preferably one or two organic radicals with the chain length described above.

According to a preferred embodiment of the present disclosure, the aqueous carrier of the two-component hair care product contains a cationic tenside which is a tetraalkylammonium compound, preferably a trimethyl alkylammonium halide, a trimethyl alkylammonium methosulfate, a N-[3-(dimethylamino)propyl]alkanamide, particularly N-[3-(dimethylamino)propyl]octadecanamide, or an esterquat. The aqueous carrier most particularly preferably contains Cetrimonium Chloride (INCI) and/or Stearamidopropyl Dimethylamine (INCI).

According to the preferred embodiment, the aqueous carrier contains a cationic tenside having formula (1) which represents a tetraalkylammonium compound, that is to say

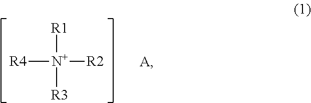

in which R1, R2, R3 and R4 each independently of each other stand for hydrogen, a methyl group, a phenyl group, a benzyl group, a saturated, branched or unbranched alkyl radical with a chain length from 12 to 30 carbon atoms, which may or may not be substituted with one or more hydroxy groups, providing that at least one of the radicals R1, R2, R3 or R4 is not hydrogen and represents a saturated, branched or unbranched alkyl radical with a chain length from 12 to 30 carbon atoms, and wherein A stands for a physiologically compatible anion, for example halides such as chloride or bromide and methosulfates.

According to a preferred embodiment of the present disclosure, the cationic tenside with formula (1) selected from the group including lauryl trimethylammonium chloride, lauryl trimethylammonium methosulfate, cetyl trimethylammonium chloride, cetyl trimethylammonium bromide, cetyl trimethylammonium methosulfate, dicetyl dimethylammonium chloride, dicetyl dimethylammonium methosulfate, tricetyl methylammonium chloride, tricetyl methylammonium methosulfate, stearyl trimethylammonium chloride, stearyl trimethylammonium methosulfate, distearyl dimethylammonium chloride, distearyl dimethylammonium methosulfate, lauryl dimethylbenzylammonium chloride, behenyl trimethylammonium chloride, behenyl trimethylammonium bromide, behenyl trimethylammonium methosulfate, dimethyl dibehenylammonium chloride, tribehenyl methyltrimonium chloride, cocotrimonium chloride, cocotrimonium methosulfate, dicocodimonium chloride, dicocodimonium methosulfate, tricocomonium chloride, tricocomonium methosulfate and mixtures thereof.

Further particularly preferred cationic tensides which may be used as contemplated herein are selected from esterquats. Particularly preferred esterquats as contemplated herein are distearoylethyl hydroxyethylmonium methosulfate and dicocoylethyl hydroxyethylmonium methosulfate. Such esterquat tensides are marketed commercially under the trade names Rewoquat®, Stepantex®, Dehyquart® and Armocare®, for example. The products Quartamin BTC 131, Armocare® VGH-70, a N,N-bis(2-palmitoyloxyethyl)dimethylammonium chloride, and Dehyquart® F-75, Dehyquart® C-4046, Dehyquart® L80, Dehyquart® F-30, Dehyquart® AU-35, Rewoquat® WE18, Rewoquat® WE38 DPG and Stepantex® VS 90 are examples of such esterquats.

According to a preferred embodiment of the two-component hair care product as contemplated herein, the two-component hair care product contains in the first component the branched or unbranched alkane in a quantity from about 20 to about 90 wt %, preferably from about 30 to about 80 wt %, more preferably from about 40 to about 70 wt %, most preferably from about 45 to about 65 wt % relative to the total weight of the first component. According to a further preferred embodiment of the present disclosure, the two-component hair care product contains the fatty alcohol in the first component in a quantity from about 5 to about 40 wt %, preferably from about 10 to about 37 wt %, more preferably from about 15 to about 34 wt %, most preferably from about 20 to about 30 wt % relative to the total weight of the first component. According to a further preferred embodiment of the two-component hair care product as contemplated herein, the two-component hair care product contains the alkylester, the alkylamide or the ether in the first component in a quantity from about 1 to about 20 wt %, preferably from about 2 to about 15 wt %, more preferably from about 3 to about 10 wt % relative to the total weight of the first component.

According to a further preferred embodiment of the present disclosure, the two-component hair care product contains water and from about 0.5 to about 20 wt %, preferably from about 1.5 to about 15 wt %, more preferably from about 2.5 to about 10 wt % cationic tenside relative to the total weight of the second component in the second component.

The ranges indicated are associated with the advantage that the solution to the problem addressed by the present disclosure can be attained.

Preferred two-component hair care products contain the first component and the second component in a weight ratio of the first component to the second component in the range from about 10 to 1 to about 1 to 10, preferably from about 5 to 1 to about 1 to 5, more preferably from about 2 to 1 to about 1 to 2, exceedingly preferably in the weight ratio about 1:1. Particularly when the components contain the constituents in the preferred quantities specified above, two-component hair care products may be prepared that are easy to dispense in metered quantities, since the abovementioned weight ratios according to this preferred embodiment are easily manageable.

In a preferred two-component hair care product, the first component is a transparent composition, and/or the second component is a transparent composition, preferably both the first and second components are transparent compositions. This preferred embodiment responds to a more demanding customer requirement.

A central aspect of the present disclosure relates to the feature according to which the hair care product as contemplated herein is provided in the form of a two-component hair care product. The two-component hair care product is prepared in such manner that the user can take both components out of an associated container. According to a preferred embodiment of the present disclosure, the first component and the second component are provided in such manner that they are separated from each other by a barrier which is substantially impermeable to liquids, in particular by at least one film. This may be designed in such manner that two adjoined sachets each contain one component. According to a preferred embodiment of the present disclosure, the first component and the second component are contained in a packaging unit in such manner that they are separated from each other by a barrier which is substantially impermeable to liquids, in particular by at least one film. The barrier which is impermeable to liquids is designed to ensure that the two components do not mix with one another until the packaging unit is opened.

The problem to be addressed by the present disclosure is further solved by the object of Claim 13. A second object of the present disclosure is therefore a method for treating hair, wherein in a first step the first component and the second component of the two-component hair care product as contemplated herein are mixed so that an emulsion is formed, in a second step the emulsion is allowed to stand for a period between about 1 second and about 30 minutes, preferably between about 20 seconds and about 20 minutes, more preferably between about 30 seconds and about 10 minutes, and in a third step following on from the second step the emulsion is applied to the keratinous fibres.

A major advantage of the present disclosure consists in the ease with which an emulsion may be formed with the aqueous carrier and the anhydrous carrier. An emulsion may be obtained quite quickly by agitation. This emulsion is then ready for use immediately and is spread on the hair as specified in the method as contemplated herein.

According to a preferred embodiment of the method as contemplated herein, the emulsion applied to the keratinous fibres is left on the keratinous fibres. According to this first variant, the product as contemplated herein is used as a leave-on product. Alternatively, according to another preferred embodiment the emulsion applied to the keratinous fibres is rinsed out of the keratinous fibres after a period of from about 20 seconds to about 5 minutes, preferably from about 45 seconds to about 2 minutes. According to this alternative, the composition as contemplated herein is used as a rinse-off product.

According to a further preferred embodiment of the present disclosure, in the first step the first component and the second component are mixed with each other at a temperature of from about 15 to about 30° C. The preparation of the emulsion is thus preferably carried out without a heating step. By virtue of the inventive formulation of the aqueous carrier and the anhydrous carrier, said carriers may be mixed form an emulsion without difficulty.

The problem to be addressed by the present disclosure is further solved by the object of Claim 15. A third object of the present disclosure is thus the use of the two-component hair care product as contemplated herein for grooming, preferably conditioning keratinous fibres, in particular human hair.

Features relating to preferred embodiments of the first object of the present disclosure which are described only with reference thereto above naturally constitute corresponding features of preferred embodiments of the second object and the third object as well.

Most particularly preferred compositions as contemplated herein comprise at least one of the following embodiments A) to I):

### A)

Two-component hair care product, comprising an anhydrous carrier as a first component and an aqueous carrier as a second component, separated from each other, the anhydrous carrier contains a) a branched or unbranched alkane with a chain length from C10 to C30, preferably from C12 to C24, more preferably from C14 to C20, b) a branched or unbranched fatty alcohol with a chain length from C10 to C22, preferably C12 to C18, more preferably from C14 to C16, and c) an ether which is an alkoxylated fatty alcohol preferably having formula HO(C₃H₆O)ₙCₘH₂ₘ₊₁, in which n is a number from about 1 to about 6, preferably from about 2 to about 4, and m is a number from about 5 to about 14, more preferably from about 6 to about 12, more preferably from about 8 to about 10,
and the aqueous carrier comprises a cationic tenside which comprises an organic radical with a linear or branched chain length from C12 to C30, preferably C14 to C26, more preferably from C16 to C22.

### B)

Two-component hair care product, comprising an anhydrous carrier as a first component and an aqueous carrier as a second component, separated from each other, the anhydrous carrier contains a) a branched or unbranched alkane with a chain length from C10 to C30, preferably from C12 to C24, more preferably from C14 to C20, b) a branched or unbranched fatty alcohol with a chain length from C10 to C22, preferably C12 to C18, more preferably from C14 to C16, and c) an alkyl ester which is a fatty acid ester that is preferably a glycerol triester which more preferably has identical or different acyl groups, wherein more preferably still the acyl groups each include 5 to 11, preferably 7 to 9, carbon atoms,
and the aqueous carrier comprises a cationic tenside which comprises an organic radical with a linear or branched chain length from C12 to C30, preferably C14 to C26, more preferably from C16 to C22.

### C)

Two-component hair care product, comprising an anhydrous carrier as a first component and an aqueous carrier as a second component, separated from each other, the anhydrous carrier contains a) a branched or unbranched alkane with a chain length from C10 to C30, preferably from C12 to C24, more preferably from C14 to C20, b) a branched or unbranched fatty alcohol with a chain length from C10 to C22, preferably C12 to C18, more preferably from C14 to C16, and c) an alkyl ester which is a fatty acid ester that is preferably a product of esterification from a C4 to C14, preferably C6 to C10, fatty acid and a C4 to C14, preferably C6 to C10, fatty alcohol,
and the aqueous carrier comprises a cationic tenside which comprises an organic radical with a linear or branched chain length from C12 to C30, preferably C14 to C26, more preferably from C16 to C22.

### D)

Two-component hair care product, comprising an anhydrous carrier as a first component and an aqueous carrier as a second component, separated from each other, the anhydrous carrier contains a) a branched or unbranched alkane with a chain length from C10 to C30, preferably from C12 to C24, more preferably from C14 to C20, b) a branched or unbranched fatty alcohol with a chain length from C10 to C22, preferably C12 to C18, more preferably from C14 to C16, and c) a N,N-dimethyl-carboxylic acid amide having a C8 to C18, preferably a C10 to C16, more preferably a C12 to C14 acyl group,
and the aqueous carrier comprises a cationic tenside which comprises an organic radical with a linear or branched chain length from C12 to C30, preferably C14 to C26, more preferably from C16 to C22.

### E)

Two-component hair care product, comprising an anhydrous carrier as a first component and an aqueous carrier as a second component, separated from each other, the anhydrous carrier contains a) a branched or unbranched alkane with a chain length from C10 to C30, preferably from C12 to C24, more preferably from C14 to C20, b) a branched or unbranched fatty alcohol with a chain length from C10 to C22, preferably C12 to C18, more preferably from C14 to C16, and c) at least one compound selected from the group including PPG-3-Caprylyl Ether (INCI), Capric/Caprylic Triglyceride (INCI), Coco Caprylate (INCI) and Dimethyl Lauramide/Myristamide (INCI),
and the aqueous carrier comprises a cationic tenside which comprises an organic radical with a linear or branched chain length from C12 to C30, preferably C14 to C26, more preferably from C16 to C22.

### F)

Two-component hair care product, comprising an anhydrous carrier as a first component and an aqueous carrier as a second component, separated from each other, the anhydrous carrier contains a) a branched or unbranched alkane with a chain length from C10 to C30, preferably from C12 to C24, more preferably from C14 to C20, b) a branched or unbranched fatty alcohol with a chain length from C10 to C22, preferably C12 to C18, more preferably from C14 to C16, and c) an alkyl ester, a N,N-dialkyl amide or an ether, wherein the total carbon atomic number from the alkyl ester, the N,N-dialkyl amide or the ether is from about 6 to about 30, preferably from about 8 to about 22, more preferably from about 10 to about 16, most preferably from about 12 to about 14,
and the aqueous carrier comprises a cationic tenside which is a tetraalkylammonium compound, preferably a trimethyl alkylammonium halide, a trimethyl alkylammonium methosulfate, a N-[3-(dimethylamino)propyl]alkanamide, particularly N-[3-(dimethylamino)propyl]octadecanamide, or an esterquat.

### G)

Two-component hair care product, comprising an anhydrous carrier as a first component and an aqueous carrier as a second component, separated from each other, the anhydrous carrier contains a) a dodecane or isododecane, b) a branched or unbranched fatty alcohol with a chain length from C10 to C22, preferably C12 to C18, more preferably from C14 to C16, and c) an alkyl ester, a N,N-dialkyl amide or an ether, wherein the total carbon atomic number from the alkyl ester, the N,N-dialkyl amide or the ether is from about 6 to about 30, preferably from about 8 to about 22, more preferably from about 10 to about 16, most preferably from about 12 to about 14,
and the aqueous carrier comprises a cationic tenside which is a tetraalkylammonium compound, preferably a trimethyl alkylammonium halide, a trimethyl alkylammonium methosulfate, a N-[3-(dimethylamino)propyl]alkanamide, particularly N-[3-(dimethylamino)propyl]octadecanamide, or an esterquat.

### H)

Two-component hair care product, comprising an anhydrous carrier as a first component and an aqueous carrier as a second component, separated from each other, the anhydrous carrier contains a) a dodecane or isododecane, b) a fatty alcohol selected from the group including Coconut Alcohol (INCI), Cetearyl Alcohol (INCI), Stearyl Alcohol (INCI), Cetyl Alcohol (INCI), Myristyl Alcohol (INCI), Lauryl Alcohol (INCI) and Decyl Alcohol (INCI), and
c) an alkyl ester, a N,N-dialkyl amide or an ether, wherein the total carbon atomic number from the alkyl ester, the N,N-dialkyl amide or the ether is from about 6 to about 30, preferably from about 8 to about 22, more preferably from about 10 to about 16, most preferably from about 12 to about 14,
and the aqueous carrier comprises a cationic tenside which is a tetraalkylammonium compound, preferably a trimethyl alkylammonium halide, a trimethyl alkylammonium methosulfate, a N-[3-(dimethylamino)propyl]alkanamide, particularly N-[3-(dimethylamino)propyl]octadecanamide, or an esterquat.

### I)

Two-component hair care product, comprising an anhydrous carrier as a first component and an aqueous carrier as a second component, separated from each other, the anhydrous carrier contains a) dodecane or isododecane in a quantity from about 20 to about 90 wt %, preferably from about 30 to about 80 wt %, more preferably from about 40 to about 70 wt %, most preferably from about 45 to about 65 wt % relative to the total weight of the first component, b) a branched or unbranched fatty alcohol with a chain length from C10 to C22, preferably C12 to C18, more preferably from C14 to C16, and c) an alkyl ester, a N,N-dialkyl amide or an ether, wherein the total carbon atomic number from the alkyl ester, the N,N-dialkyl amide or the ether is from about 6 to about 30, preferably from about 8 to about 22, more preferably from about 10 to about 16, most preferably from about 12 to about 14,
and the aqueous carrier comprises a cationic tenside which is a tetraalkylammonium compound, preferably a trimethyl alkylammonium halide, a trimethyl alkylammonium methosulfate, a N-[3-(dimethylamino)propyl]alkanamide, particularly N-[3-(dimethylamino)propyl]octadecanamide, or an esterquat.

### EXAMPLES

The following formulations can be prepared:

### Example 1: Anhydrous Phase

| Constituent | Content in wt % |
|---|---|
| Isododecane | 63.6 |
| Lauryl alcohol | 5.0 |
| Decyl alcohol | 5.0 |
| Myristyl alcohol | 5.0 |
| Cetearyl alcohol | 15.0 |
| Caprylic/Capric Triglyceride | 2.5 |
| PPG-3-Caprylyl Ether (INCI) | 2.5 |
| Fragrance | 1.4 |

### Example 2: Anhydrous Phase

| Constituent | Content in wt % |
|---|---|
| Isododecane | 66.1 |
| Cetyl alcohol | 5.0 |
| Coconut alcohol | 20.0 |
| Dimethyl Lauramide/Myristamide (INCI) | 2.5 |
| Caprylic/Capric Triglyceride | 2.5 |
| PPG-3-Caprylyl Ether (INCI) | 2.5 |
| Fragrance | 1.4 |

### Aqueous Phase

| Constituent | Content in wt % |
|---|---|
| Water | 96.8 |
| Sodium benzoate | 0.5 |
| Lactic acid | 0.4 |
| Stearamidopropyl Dimethylamine (INCI) | 0.8 |
| Cetrimonium chloride | 1.5 |

The anhydrous phase of Example 1 and the anhydrous phase of Example 2 are each mixed with the aqueous phase. The two phases were mixed with each other in a weight ratio of 1:1 simply by shaking by hand and formed an emulsion.

The hair care products obtained in this way are applied to dry or damp hair, and rinsed out again with lukewarm water at 20° C. after an application time of 5 minutes. The hair treated in this way was soft and had a pleasing shine. When the hair was subsequently dried, it was smooth and did not curl (no frizz).

In a variation of the hair treatment method as contemplated herein, the hair care products obtained in this way were sprayed onto the dry hair using a pump sprayer. The hair treated in this way was soft, had a pleasing shine, and did not curl (no frizz).

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Two-component hair care product, comprising an anhydrous carrier as a first component and an aqueous carrier as a second component, separated from each other, wherein the anhydrous carrier contains
a) a branched or unbranched alkane with a chain length from C10 to C30,
b) a branched or unbranched fatty alcohol with a chain length from C10 to C22, and
c) an alkyl ester, a N,N-dialkyl amide or an ether, wherein the total carbon atomic number of the alkyl ester, the N,N-dialkyl amide or the ether is from 6 to 30,
and the aqueous carrier comprises a cationic tenside consisting of an organic radical with a linear or branched chain length of C12 to C30.

2. Two-component hair care product according to claim 1, wherein the two-component hair care product contains as constituent c) an ether which is an alkoxylated fatty alcohol.

3. Two-component hair care product according to claim 1, wherein the two-component hair care product contains as constituent c) an alkyl ester which is a fatty acid ester, or wherein the two-component hair care product contains as constituent c) an alkyl ester.

4. Two-component hair care product according to claim 1, wherein the two-component hair care product contains as constituent c) a N,N-dialkyl amide which is a fatty acid amide.

5. Two-component hair care product according to claim 1, wherein the branched or unbranched alkane is dodecane or isododecane.

6. Two-component hair care product according to claim 1, wherein the branched or unbranched fatty alcohol is selected from the group consisting of Coconut Alcohol (INCI), Cetearyl Alcohol (INCI), Stearyl Alcohol (INCI), Cetyl Alcohol (INCI), Myristyl Alcohol (INCI), Lauryl Alcohol (INCI) and Decyl Alcohol (INCI).

7. Two-component hair care product according to claim 1, wherein the component c) is selected from the group consisting of PPG-3 Caprylyl Ether (INCI), Capric/Caprylic Triglyceride (INCI), Dimethyl Lauramide/Myristamide (INCI) and Coco Caprylate (INCI).

8. Two-component hair care product according to claim 1, wherein the cationic tenside is a tetraalkylammonium compound.

9. Two-component hair care product according to claim 1, wherein the two-component hair care product contains in the first component the branched or unbranched alkane in a quantity from 20 to 90 wt % relative to the total weight of the first component, the fatty alcohol in a quantity from 5 to 40 wt % relative to the total weight of the first component, and the alkyl ester, the alkyl amide or the ether in a quantity from 1 to 20 wt % relative to the total weight of the first component, and/or wherein the two-component hair care product contains in the second component water and, relative to the total weight of the second component, 0.5 to 20 wt %.

10. Two-component hair care product according to claim 1, wherein the weight ratio of the first component to the second component is from 10 to 1 to 1 to 10.

11. Two-component hair care product according to claim 1, wherein the first component is a transparent composition, and/or the second component is a transparent composition.

12. Two-component hair care product according to claim 1, wherein the first component and the second component are provided in such manner that they are separated from each other by a barrier which is substantially impermeable to liquids, in particular by at least one film, or wherein the first component and the second component are contained in a packaging unit in such manner that they are separated from each other by a barrier which is substantially impermeable to liquids, in particular by at least one film.

13. Method for treating hair, wherein in a first step the first component and the second component of the two-component hair care product according to claim 1 are mixed so that an emulsion is formed, in a second step the emulsion is allowed to stand for a period between 1 second and 30 minutes, and in a third step following on from the second step the emulsion is applied to the keratinous fibres.

14. Method according to claim 13, wherein the emulsion applied to the keratinous fibres is left on the keratinous fibres, or wherein the emulsion applied to the keratinous fibres is rinsed out of the keratinous fibres after a period of 20 seconds to 5 minutes.

15. Use of the two-component hair care product according to claim 1 to groom, preferably to condition, keratinous fibres.
